(19) United States
(12) Patent Application Publication
(10) Pub. No.: US 2007/0272157 A1
(43) Pub. Date: Nov. 29, 2007

(54) Identification System, Especially For Small Animals

(76) Inventors: Eitan Uner, Moledet (IL); Tsafrir Pinski, Afikim (IL)

Correspondence Address: FRISHAUF, HOLTZ, GOODMAN & CHICK, PC, 220 Fifth Avenue, 16TH Floor, NEW YORK, NY, 10001-7708, US

(21) Appl. No.: 11/660,190

(22) PCT Filed: Aug. 25, 2005
(86) PCT No.: PCT/IL05/00920

§ 371 Date: Jul. 18, 2007

(30) Foreign Application Priority Data
Aug. 31, 2004 (IL) 163854

(51) Int. Cl. A01J 99/00; G01S 13/75

(52) U.S. Cl. 119/14.04; 119/14.01; 340/286.01

(57) ABSTRACT

An identification system, especially for identifying small animals that are carrying ID-transponders is disclosed. The system id comprised of a hanger that is installed above the track where an animal is passing or being passed; a flexible sheet that is handed down from the hanger and while passing through the track the animal pushes the flexible sheet in a way that the flexible sheet touches the animal or at least the animal passes near the flexible sheet; an antenna that is installed in the flexible sheet; a transceiver that is using the antenna to communicate with an ID-transponder that is carried by the passing animal in order to identify the animal; and a controller, the controller is operative for receiving the animal identification and capable to store, process and display the identifications.

### FIELD OF THE INVENTION

The present invention relates to the field of identifying animals while passing through a checkpoint, especially for identifying small animals.

### BACKGROUND OF THE INVENTION

The use of transponders or other transmitters to locate and/or identify animals is well known. More particularly the use of transponders in identifying individual cows during a milking operation has been much discussed in the dairy farm management literature and has been widely employed. Cows may be identified by attaching transmitters to collars or, alternatively, encasing transponders in ear tags, which are attached to cartilaginous tissue of the ear. Suitable receivers can be fixed in a milking stall to read the identification data signals of a cow as it enters the stall or as it is being milked. Such identification systems are described or illustrated inter-alia in U.S. Pat. No. 5,996,529 to Sissom et al, U.S. Pat. No. 6,279,507 to Van Der Lely et al, U.S. Pat. No. 6,394,028 to Birk and U.S. Pat. No. 6,445,296 to Sadler.

Identification methods are necessary in dairy farm management because accurately measuring and recording the productivity of an individual animal within a herd is important. Known systems identify an animal being milked and measure the amount of milk that the animal produces. The systems record that data, typically through microprocessors, so that the data may later be analyzed.

Generally, prior art identification systems are adequate for large animals, such as cows. Because cows are large animals and the milking stalls are far enough away from each other, transmitted identification data signals related to a particular bovine can be read without interference from the signals of other animals in the milking area at the same time. For smaller animals, typically, but without being limiting, goats and sheep, such interference can be problematic. Additionally, in order to reliably detect the tags, which may be at varying distances from the antennas, the frequency needs to be relatively high. Often, however, FCC regulations prohibit use of sufficiently high frequencies.

Smaller animals, such as sheep and goats, are not generally milked in individual stalls. They are crowded together, more or less one next to another, during the milking operation. Signals from identification transponders of the closely positioned animals may interfere with each other, making identification of individual animals difficult. Therefore, the identification can be done while the animal is passing through a lane, e.g. lane that leads the animal to the milking point. The present invention provides a system for identify animals during passage through a checkpoint.

### SUMMARY OF THE INVENTION

The present invention is an identification system, especially for identifying small animals that are carrying ID-transponders.

According to the teachings of the present invention there is provided an identification system, especially for identifying small animals that are carrying ID-transponders. The system comprising:
a hanger that is installed above the track where an animal is passing or being passed;
a flexible sheet that is handed down from the hanger and while passing through the track the animal pushes the flexible sheet in a way that the flexible sheet touches the animal or at least the animal passes near the flexible sheet;
an antenna that is installed in the flexible sheet;
a transceiver that is using the antenna to communicate with an ID-transponder that is carried by the passing animal in order to identify the animal; and
a controller, the controller is operative for receiving the animal identification and capable to store, process and display the identifications.

According to further features in the described preferred embodiment the identification system is provided wherein the flexible sheet is pivotally joined to the hanger.

According to another preferred embodiment, the identification system is provided wherein the antenna is floating in a fitted space that is located inside the flexible sheet.

According to another preferred embodiment, the identification system is provided wherein the system is installed in a rotary milking-parlor.

According to another preferred embodiment, the identification system is provided wherein the system is installed in a parallel milking-parlor.

According to another aspect of the present invention, it is provided a rotary milking-parlor with the identification system as defined, wherein the identification system is installed in a way that the flexible sheet is hanged in the animals' heads passage and while the milking-parlor is rotating the flexible sheet is pushed by each animal's head, enabling the identification system to communicate with the animal's transponder in order to identify each animal in accordance with the stall where the animal is located.

According to a preferred embodiment of the rotary milking-parlor, it is provided wherein it further includes a plurality of stall-ID-transponders, each of the stall-ID-transponders is installed in a stall, enabling the identification system to communicate with the stall-ID-transponders and to identify the animal and the stall where the animal is located, at once.

According to another aspect of the present invention, it is provided a parallel milking-parlor with an identification system as defined, wherein the identification system is further includes a convey means that capable to convey the flexible sheet along the stalls of the parallel milking-parlor—when animals are located in the stalls—in a way that the flexible sheet is slides on the animals' body in order to communicate with the transponder that is carried by the animal and wherein the controller receives from the transceiver the animal's identification and the stall location from the convey means, enabling the identification system to identify each animal in accordance with the stall where the animal is located.

According to a preferred embodiment of the parallel milking-parlor, it is provided wherein it includes a plurality of stall-ID-transponders, each of the stall-ID-transponders is installed in a stall, enabling the identification system to communicate with the stall-ID-transponders and to identify the animal and the stall where the animal is located, at once.

According to another aspect of the present invention it is provided a system for identifying a ruminant being milked, wherein the system including:
a transmitter attached to a ruminant and operable to transmit a predefined signal identifying the ruminant to which the transmitter is attached; and
an identifying system as previously defined.

By a preferred embodiment of the present invention the ruminant identification system is provided wherein the transmitter is adapted to be swallowed by a ruminant and remains in the ruminant stomach.

By another preferred embodiment of the present invention, the ruminant identification system is provided wherein the transmitter is a transponder.

By another preferred embodiment of the present invention the ruminant identification system is provided wherein the system further includes a stationary interrogation unit; this unit includes a transmitter and a receiver.

By yet another preferred embodiment of the present invention, the ruminant identification system is provided wherein the transponder is adapted for attachment to the ruminant.

### BRIEF DESCRIPTION OF THE FIGURES

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the figures:

FIG. 1 illustrates an embodiment of the system, according to the present invention, installed in a rotary milking-parlor.

FIG. 2 illustrates an embodiment of the system, according to the present invention, installed in a parallel milking-parlor.

FIG. 3 illustrates an embodiment of the system, according to the present invention, installed in a passing gate.

### DESCRIPTION OF THE PREFERED EMBODIMENTS

The present invention is an identifying system, especially for small animals and ruminants.

Ruminants are led crowded into a milking-parlor, for milking, eating etc. Usually, each ruminant carries on its' body or inside its' stomach a transponder or alike for identification. The milking-parlor contains stalls and each ruminant is directed into a stall. The present invention is dedicated to identify each ruminant in the milking-parlor from a very close range. The very close range enables low power transmission in order to prevent interfere transmissions from the neighbor animals.

The present invention comprised of a flexible sheet that contains an antenna, which hanged on a hanger in the track where the ruminant is passes or being passed. Part of the ruminant's body pushes the flexible sheet and creates a contact that enables a very close communication with the transponder that is carried by the ruminant. If the ruminant dose not pushes the flexible sheet, at least the ruminant passes very close to it.

The antenna is installed inside the flexible sheet and enables a transceiver to communicate with the transponder, which is carried by the ruminant, from a close range. The flexible sheet can be hanged in a rotary milking-parlor and the ruminants push it while the parlor is rotating. In a parallel milking-parlor the flexible sheet is conveyed along the stalls and slides on the animals that are located inside. The stall, in which the ruminant is located, can be recognized by the parlor position indication or by communicating with a transponder that can be attached to each stall.

The principles and operation of the identification system according to the present invention may be better understood with reference to the drawing and the accompanying description.

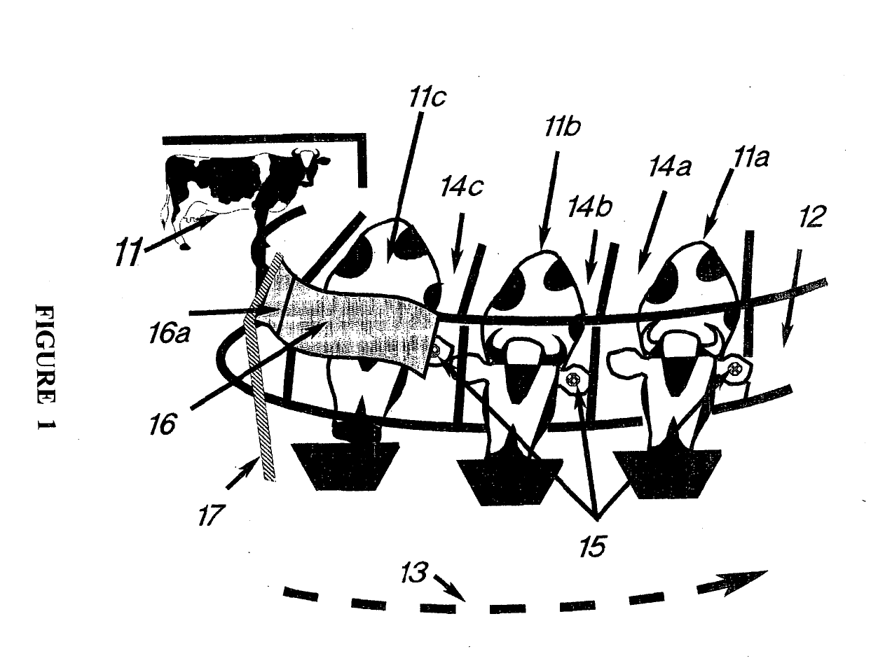

Referring now to the drawing, FIG. 1 illustrates an embodiment of the system, according to the present invention, installed in a rotary milking-parlor. The ruminants 11 are leaded to enter into the rotary milking-parlor 12 that—in this case—rotates anticlockwise 13. During the parlor rotation each ruminant occupies a stall when entering, first ruminant 11a occupies the first stall 14a, second ruminant 11b occupies the second stall 14b and so on. Each ruminant 11 has a transponder 15 for identification that is attached to its' ear. The flexible sheet 16 with the antenna inside it is hanged via an axis 16a onto a hanger 17. When the next ruminant 11c enters to the next stall 14c, the milking-parlor 12 is rotating—with the ruminants on it—and the head of the ruminant 11c pushes the flexible sheet 16, which slides on the ruminant's head.

The system uses the antenna that is located inside the flexible sheet 16 to communicate with the transponder 15 that is carried by the ruminant 11c in order to identify the ruminant 11c in the stall 14c and stores the information for further processing. The system recognizes the stall 14c either by having a position indication of the milking-parlor 12 or by communicating with a transponder (not shown) that can be attached to each stalls.

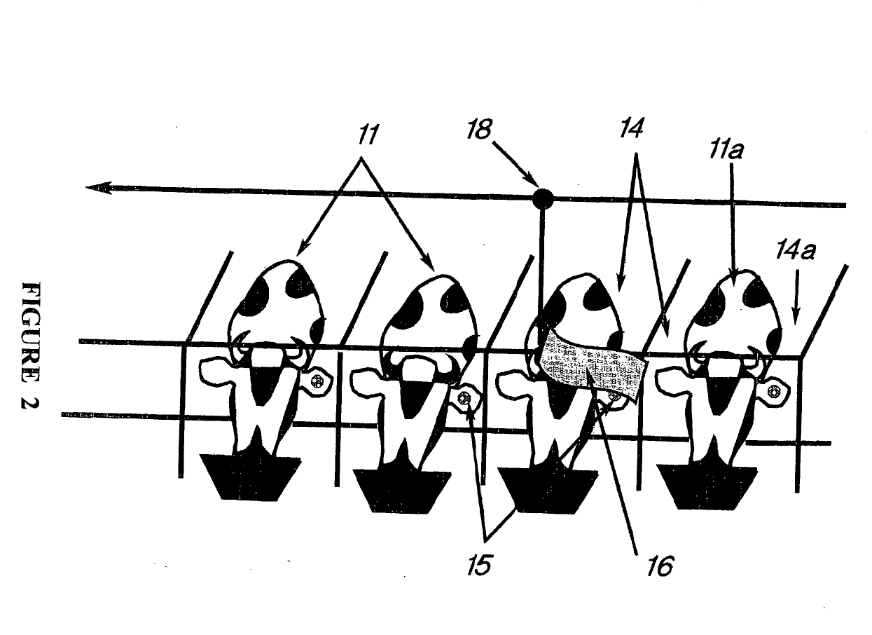

FIG. 2 illustrates an embodiment of the system, according to the present invention, installed in a parallel milking-parlor. In this case, the ruminants 11 are leaded crowded into a battery of stalls 14. When the ruminants 14 are occupying the stalls 14 the convey means 18 conveys the flexible sheet 16 along the stalls 14 in a way that the flexible sheet 16 slides on the heads of whole ruminants. While the flexible sheet 16 is passing on specific ruminant's head 11a, the system communicates with the transponder 15 that is carried by the ruminant 11a and identifies it in stall 14a that is recognized by the position of the convey means 18. A controller stores the information for further processing.

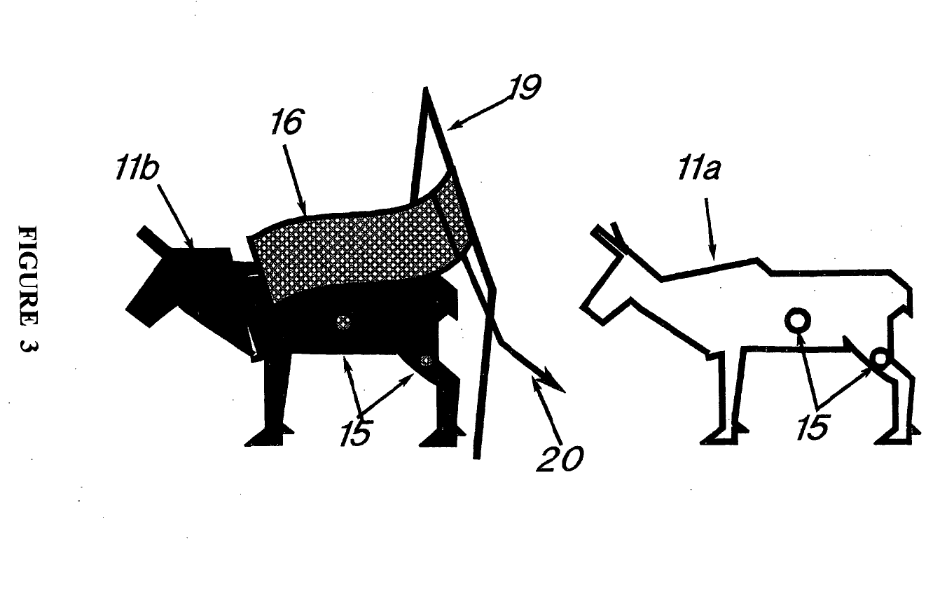

FIG. 3 illustrates an embodiment of the system, according to the present invention, installed in a passing gate. Ruminants 11a & 11b, with transponders 15 on theirs bodies or in theirs stomach, are passing through a frame-head 19 on which a flexible sheet 16 with antenna is hanged down from top of the frame-head 19.

The ruminant 11b is pushing the flexible sheet 16, which touches the body of the ruminant 11b and is very close to the carried transponder 15. An antenna (not shown) is installed in the flexible sheet 16 and connected via cable 20 to a transceiver (not shown) enables the transceiver to communicate with the transponder 15 from a very close range and enables the system to identify each passing ruminant.

Although the invention has been described in conjunction with specific embodiments thereof, it is evident that many alternatives, modifications and variations will be apparent to those skilled in the art, accordingly, it is intended to embrace all such alternatives, modifications and variations that fall within the spirit and broad scope of the appended claims.

What is claimed is:

1. An identification system for identifying animals that are carrying ID-transponders, said system comprising:
a hanger that is installed above the track where an animal is passing or being passed;
a flexible sheet that is handed down from said hanger and while passing through said track said animal pushes said flexible sheet in a way that said flexible sheet touches said animal or at least said animal passes near said flexible sheet;
an antenna that is installed in said flexible sheet;
a transceiver that is using said antenna to communicate with an ID-transponder that is carried by said animal in order to identify said animal; and
a controller, said controller is operative for receiving said animal's identification and capable to store, process and display said identification.

2. The identification system of claim 1, wherein said flexible sheet is pivotally joined to said hanger.

3. The identification system of claim 1, wherein said antenna is floating in a fitted space that is located inside said flexible sheet.

4. The identification system of claim 1, wherein said identification system is installed in a rotary milking-parlor.

5. The identification system of claim 1, wherein said identification system is installed in a parallel milking-parlor.

6. A rotary milking-parlor with an identification system as defined in claim 1, wherein said identification system is installed in a way that said flexible sheet is hanged in the animals' heads passage and while said milking-parlor is rotating said flexible sheet is pushed by each animal's head, enabling said identification system to identify each animal in accordance with the stall where said animal is located.

7. The rotary milking-parlor of claim 6, further includes a plurality of stall-ID-transponders, each of said stall-ID-transponders is installed in a stall, enabling said identification system to communicate with said stall-ID-transponders and to identify the animal and the stall where said animal is located, at once.

8. A parallel milking-parlor with an identification system as defined in claim 1, wherein said identification system is further includes a convey means that capable to convey said flexible sheet along the stalls of said parallel milking-parlor—when animals are located in said stalls—in a way that said flexible sheet is slides on the animals' body in order to communicate with the each transponder that is carried by the animal and wherein said controller receives from said transceiver said animal identification and said stall location from said convey means, enabling said identification system to identify each animal in accordance with the stall where said animal is located.

9. The parallel milking-parlor of claim 8, further includes a plurality of stall-ID-transponders, each of said stall-ID-transponders is installed in a stall, enabling said identification system to communicate with said stall-ID-transponders and to identify the animal and the stall where said animal is located, at once.

10. A system for identifying a ruminant being milked, said system including:
a transmitter attached to a ruminant and operable to transmit a predefined signal identifying the ruminant to which said transmitter is attached; and
an identifying system as defined in claim 6.

11. The system of claim 10, wherein said identifying system is as defined in claim 8.

12. The system of claim 10, wherein said transmitter is adapted to be swallowed by a ruminant and remains in said ruminant stomach.

13. The system of claim 10 wherein said transmitter is a transponder.

14. The system of claim 13 wherein said system further includes a stationary interrogation unit, said unit including a transmitter and a receiver.

15. The system of claim 13 wherein said transponder is adapted for attachment to the ruminant.

16. The system of claim 13, wherein said transponder is adapted to be swallowed by a ruminant and remains in said ruminant stomach.
